(19) United States
(12) Patent Application Publication
(10) Pub. No.: US 2024/0360591 A1
(43) Pub. Date: Oct. 31, 2024

(54) METHODS FOR SELECTING EXCIPIENTS AND FOR PREPARING HIGH CONCENTRATION MONOCLONAL ANTIBODY FORMULATIONS AND OTHER FORMULATIONS COMPRISING PROTEINS

(71) Applicant: Regeneron Pharmaceuticals, Inc., Tarrytown, NY (US)

(72) Inventors: Xiaolin Tang, Old Tappan, NJ (US); Leonid Breydo, Bronx, NY (US)

(21) Appl. No.: 18/645,694

(22) Filed: Apr. 25, 2024

Related U.S. Application Data
(60) Provisional application No. 63/541,455, filed on Sep. 29, 2023.
(60) Provisional application No. 63/461,683, filed on Apr. 25, 2023.

(51) Int. Cl. C40B 30/10; G01N 33/68

(52) U.S. Cl. CPC C40B 30/10; G01N 33/6854

(57) ABSTRACT

The present inventions relate to methods of preparing highly concentrated monoclonal antibody (mAb) formulations, including screening and selection of excipients to include in mAb formulation. Excipients include amino acids, salts and polyols.

### CROSS REFERENCE TO RELATED APPLICATIONS

This application claims the benefit of and priority to U.S. Provisional Application No. 63/541,455 filed on Sep. 29, 2023 and U.S. Provisional Application No. 63/461,683 filed on Apr. 25, 2023. The above-referenced applications are incorporated herein by reference in their entirety.

### FIELD OF THE INVENTIONS

The present inventions relate to methods of preparing highly concentrated formulations of proteins of interest, including Fc-containing proteins (such as monoclonal antibodies (mAb)). The methods include screening and selection of excipients to include in such formulations.

### BACKGROUND OF THE INVENTIONS

Fc-containing proteins, such as antibodies, are an important therapeutic product in healthcare today. The extensive use of monoclonal antibody therapies has made highly concentrated formulations desirable for later dilution to final dosage forms.

High concentration monoclonal antibody formulations present quality challenges for manufacturing, storage and administration. High concentration causes high viscosity and increases antibody aggregation through protein-protein interactions (PPI).

Excipients and production methodologies play important roles in the characteristics of high concentration monoclonal antibody preparations. Excipients include amino acids, salts and polyols.

### SUMMARY OF THE INVENTIONS

The inventions provide methods for selecting excipients for inclusion in a high concentration Fc-containing protein formulations; monoclonal antibodies are a type of Fc-containing protein and serve as an example.

Monoclonal antibody formulations can be produced by methods comprising the steps of (a) combining at least one excipient with a monoclonal antibody preparation; (b) conducting a controlled nucleating cycle on the monoclonal antibody preparation from step (a); (c) drying the monoclonal antibody preparation from step (b) under a vacuum; (d) stoppering with and/or without a vacuum; and (e) evaluating excipient performance. The methods can avoid using secondary drying. Annealing also can be omitted, if desired.

The methods can screen and select excipients selected from the group consisting of amino acids, salts, polyols and combinations thereof. The excipient can be a polyol selected from the group consisting of sucrose, trehalose dihydrate, sorbitol, glycerol and combinations thereof. The excipient can be a salt selected from the group consisting magnesium chloride, sodium sulphate, ammonium sulphate, sodium bromide, sodium chloride, calcium chloride, sodium perchlorate and combinations thereof. The excipient can be an amino acid selected from the group consisting of sodium glutamate, glutamate, alanine, proline, glycine, lysine, phenylalanine, methionine, isoleucine, threonine, valine, serine, asparagine, histidine arginine hydrochloride, arginine glutamate, and combinations thereof. Monoclonal antibody preparations can comprise according to the methods of the inventions at least one excipient selected from the group consisting of (i) at least one polyol selected from the group consisting of sucrose, trehalose dihydrate, sorbitol, glycerol and combinations thereof; (ii) at least one salt selected from the group consisting magnesium chloride, sodium sulphate, ammonium sulphate, sodium bromide, sodium chloride, calcium chloride, sodium perchlorate and combinations thereof; and (iii) at least one amino acid selected from the group consisting of sodium glutamate, glutamate, alanine, proline, glycine, lysine, phenylalanine, methionine, isoleucine, threonine, valine, serine, asparagine, histidine arginine hydrochloride, arginine glutamate, and combinations thereof. Monoclonal antibody preparations can comprise according to the methods of the inventions one or more of a salt and an amino acid as excipients; one or more of a salt and a polyol as excipients; one or more of amino acid and a polyol as excipients or one or more of an amino acid, a salt and a polyol as excipients. Monoclonal antibody preparations comprising one or more excipients selected according to the methods of the inventions also are provided.

### BRIEF DESCRIPTION OF THE FIGURES

FIG. 1 is a bar graph showing reconstitution time and viscosity (cP). Test excipients (amino acids, salts and polyols) are at a concentration of 150 mM and evaluated using a monoclonal antibody at 190 mg/ml.

FIG. 2 depicts data showing the effects of reconstitution excipients on aggregation (Δ% HMW).

FIG. 3 depicts data from excipient screening (amino acids and salts) of a mAb preparation at 220 mg/ml. Reconstitution time and viscosity (cP) are depicted.

FIG. 4 depicts data from viscosity screening of single excipients vs combinations of excipients at a 1:1 ratio. The monoclonal antibody was present at 220 mg/ml.

FIG. 5 depicts the effect of pH on viscosity (cP) using a monoclonal antibody present at a concentration of 220 mg/ml.

FIG. 6 is a bar graph depicting the viscosity of reconstituted solutions of a monoclonal antibody at a concentration of 250 mg/ml with potential viscosity reducing excipients at 75 mM and 150 mM. WFI stand for water for injection (no excipient).

FIG. 7 is a bar graph depicting the viscosity of reconstituted solutions of a monoclonal antibody at a concentration of 200 mg/ml with potential viscosity reducing excipients at 75 mM and 150 mM. WFI stand for water for injection (no excipient).

### DETAILED DESCRIPTION OF THE INVENTIONS

Unless defined otherwise, all technical and scientific terms used herein have the same meaning as commonly understood by one of ordinary skill in the art to which this invention belongs.

### Definitions

The term “about” in the context of numerical values and ranges refers to values or ranges that approximate or are close to the recited values or ranges such that the invention can perform as intended, such as having a desired rate, amount, density, degree, increase, decrease, percentage, value or presence of a form, variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

The term “stoppering” refers to a function used in the lyophilization process. Lyophilization vials have stoppers on top of them for control. When a lyophilization stopper is not fully inserted in a vial, there is gap between the stopper and the mouth of the vial that allows for lyophilization to occur. Automated lyophilizers have a ‘stoppering’ function where a plate can push all the stoppers in. After this stoppering process, there is no gap between the stopper and the mouth of the vial and the vial is sealed from the atmosphere. Stoppering can be performed either while a lyophilizer is still under vacuum (vacuum stoppering) or after the chamber is filled with nitrogen gas.

“Protein of interest” or “polypeptide of interest” (POI) can have any amino acid sequence, and includes any protein, polypeptide, or peptide that is desired to be expressed. Included are, but not limited to, viral proteins, bacterial proteins, fungal proteins, plant proteins and animal (including human) proteins. Protein types can include, but are not limited to, antibodies, receptors, Fc-containing proteins, trap proteins (including mini-trap proteins), fusion proteins, antagonists, inhibitors, enzymes (such as those used in enzyme replacement therapy), factors, repressors, activators, ligands, reporter proteins, selection proteins, protein hormones, protein toxins, structural proteins, storage proteins, transport proteins, neurotransmitters and contractile proteins. Derivatives, components, domains, chains and fragments of the above also are included. The sequences can be natural, semi-synthetic or synthetic. A protein of interest is encoded by a “gene of interest” (GOI).

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cystine disulfide bond, and the two heavy chains are bound to each other via two cystine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014). Antibodies are often used as therapeutic biomolecules.

An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10.9 M, at least 10⁻¹⁰ M; at least 10⁻¹¹ M; or at least 10⁻¹² M, as measured by surface plasmon resonance, for example, BIACORE™ or solution-affinity ELISA.

The antibodies can be based upon all major antibody classes, namely IgG, IgA, IgM, IgD and IgE. IgG is a preferred class, and includes subclasses IgG1 (including IgG1λ and IgG1κ), IgG2, IgG3, and IgG4. Antibodies include a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F (ab′)2 fragment, an IgD antibody, an IgE antibody, an IgM antibody, an lgG antibody, an IgG1 antibody, an IgG2 antibody, an IgG3 antibody, or an IgG4 antibody.

The antibody can be an IgG1 antibody. The antibody can be an IgG2 antibody. The antibody can be an IgG3 antibody. The antibody is an IgG4 antibody. The antibody can be a chimeric IgG2/IgG4 antibody. The antibody can be a chimeric IgG2/IgG1 antibody. The antibody can be a chimeric IgG2/IgG1/IgG4 antibody. Derivatives, components, domains, chains and fragments of the above also are included.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen- binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen- binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FRI, CDRI, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include, but are not limited to: antibody fragments (for example, ScFv-Fc, dAB-Fc, half antibodies), multispecifics (for example, IgG-ScFv, IgG-dab, ScFV-Fc-ScFV, tri-specific) and Fc-Fusion Proteins (for example, Fc-Fusion (N-terminal), Fc-fusion (C-terminal), mono Fc-fusion, bi-specific Fc-fusion).

The phrase “Fc-containing protein” includes antibodies, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

Fc-containing proteins can comprise modifications in immunoglobulin domains, including where the modifications affect one or more effector function of the binding protein (for example, modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V2591), and a 308F (for example, V308F) modification; a 433K (for example, H433K) and a 434 (for example, 434Y) modification; a 252, 254, and 256 (for example, 252Y, 254T, and 256E) modification; a 250Q and 428L modification (for example, T250Q and M428L); a 307 and/or 308 modification (for example, 308F or 308P).

Some recombinant Fc-containing proteins contain receptors or receptor fragments, ligands or ligand fragments that have cognate binding partners in biological systems, and include “Receptor Fc-fusion proteins,” which refer to recombinant molecules that contain a soluble receptor fused to an immunoglobulin Fc domain.

“Fc-fusion proteins” comprise part or all of two or more proteins, one of which is an Fc portion of an immunoglobulin molecule, that are not fused in their natural state. Fc-fusion proteins include Fc-Fusion (N-terminal), Fc-Fusion (C-terminal), Mono Fc-Fusion and Bi-specific Fc-Fusion. Preparation of fusion proteins comprising certain heterologous polypeptides fused to various portions of antibody-derived polypeptides (including the Fc domain) has been described, for example, by Ashkenazi et al., Proc. Natl. Acad. Sci USA 88:10535-39 (1991); Byrn et al., Nature 344:677-70, 1990; and Hollenbaugh et al., “Construction of Immunoglobulin Fusion Proteins”, in Current Protocols in Immunology, Suppl. 4, pages 10.19.1-10.19.11 (1992). “Receptor Fc-fusion proteins” comprise one or more of one or more extracellular domain(s) of a receptor coupled to an Fc moiety, which can comprise a hinge region followed by a CH2 and CH3 domain of an immunoglobulin. The Fc-fusion protein can contain two or more distinct receptor chains that bind to a single or more than one ligand(s). Some receptor Fc-fusion proteins may contain ligand binding domains of multiple different receptors. Receptor Fc-fusion proteins are also referred to as “traps,” “trap molecules” or “trap proteins.” For example, such trap proteins include an IL-1 trap (for example, Rilonacept, which contains the IL-IRAcP ligand binding region fused to the IL-1R1 extracellular region fused to Fc of hIgGI; see U.S. Pat. No. 6,927,044, or a VEGF Trap (for example, Aflibercept, which contains the Ig domain 2 of the VEGF receptor FItl fused to the Ig domain 3 of the VEGF receptor FIkl fused to Fc of hIgG1; for example, SEQ ID NO: 1. See U. S. Pat. Nos. 7,087,411 and 7,279,159.

Rilonocept and aflibercept are examples of marketed trap proteins that antagonize IL1R (see US Pat. No. 7,927,583) and VEGF (see US Pat. No. 7,087,411), respectively. Other recombinant Fc-containing proteins include those recombinant proteins containing a peptide fused to an Fc domain. Recombinant Fc-containing proteins are described in C. Huang, “Receptor-Fc fusion therapeutics, traps, and MFMETIBODY technology,” 20(6) Curr. Opin. Biotechnol. 692-9 (2009).

There also are proteins that lack Fc portions, such as recombinantly produced enzymes and mini-traps, also can be used according to the inventions. Mini-traps are trap proteins that use a multimerizing component (MC) instead of an Fc portion, and are disclosed in U.S. Pat. Nos. 7,279,159 and 7,087,411. Derivatives, components, domains, chains and fragments of the above also are included.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

### Description

The inventions provide methods for selecting excipients for inclusion in a high concentration Fc-containing protein formulations; monoclonal antibodies are a type of Fc-containing protein and serve as an example.

High concentration mAb formulations required for high dose subcutaneous (SC) administration are subject to high viscosity and protein aggregation. To develop an optimized high concentration mAb formulation, screening of excipients, pH and buffers are often required. However, this screening can be difficult as protein solutions without excipients at concentrations above 200 mg/ml can reach a gelling point.

The present inventions utilize lyophilization for screening formulations of high concentration antibodies to select excipients and excipient combinations for use with monoclonal antibodies. Lyophilized mAb can be reconstituted to the desired protein concentration with any excipient and buffer combination screened and selected according to the inventions. Such screened and selected excipients are useful for high concentration monoclonal antibody preparations.

The inventions are further described by the following Examples, which are supportive and illustrative of the inventions, but do not limit the inventions in any manner.

### Example 1—The Effects of Different Lyophilization Methods on Reconstitution Time

A monoclonal antibody was dialyzed into water at moderate concentration (about 100 mg/ml) and a pre-lyophilization buffer was added. The resulting solution was lyophilized in 6R vials (1.5 ml in each vial) and reconstituted with the desired screening buffer to required protein concentration (for example, about 200 mg/ml). The pre-lyophilization buffer and lyophilization methods were optimized to minimize both reconstitution time and presence of high molecular weight (HMW) species. This method was used to screen different reconstitution buffers to minimize solution viscosity while maintaining other parameters described above in acceptable range.

Table 1 below shows the effect of vacuum stoppering on annealing and controlled nucleation lyophilization in terms of reconstitution time. Convention lyophilization was used as a control.

TABLE 1

| | Control | | | | |
|---|---|---|---|---|---|
| | Lyophilization | | | Controlled | |
| | Cycle | Annealing | | Nucleation | |
| | Vacuum stoppered | | | | |
| | No | Yes | No | Yes | No |
| Concentration | 90 | 190 | 90 | 190 | 190 |
| after | | | | | |
| reconstitution | | | | | |
| (mg/ml) | | | | | |
| Reconstitution | 55.0 | 80.0 | 54.5 | 18.0 | 51.0 |
| time with | | | | | |
| water (minutes): | | | | | |
| Δ% high molecular | 0.5 | 0.8 | 0.6 | 0.4 | — |
| weight (HMW) | | | | | |

Lyophilization of a monoclonal antibody sample in water resulted in a high level of HMW species. Adding buffer and sucrose (5 mM histidine, 1% sucrose, pH 5.9) or 1% sucrose alone decreased HMW species (less aggregation) to acceptable level. Lyophilization cycle was initially optimized by eliminating secondary drying that was shown to be unnecessary. Aggregation tends to increase with exposure to drying and thus eliminating secondary drying lessens aggregation.

To decrease the reconstitution time, controlled nucleation and annealing as well as vacuum stoppering were tested. Controlled nucleation at −5° C. combined with vacuum stoppering resulted in the shortest reconstitution time and acceptable HMW levels. Annealing was less effective in decreasing reconstitution time. Controlled nucleation optionally can use temperature controlled freezing to reduce supercooling, for example.

### Example 2—Screening of Different Viscosity Reducers

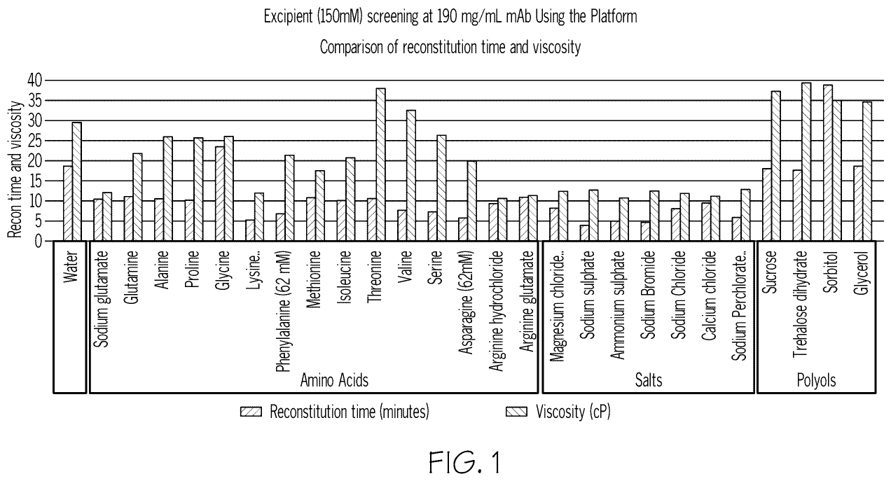

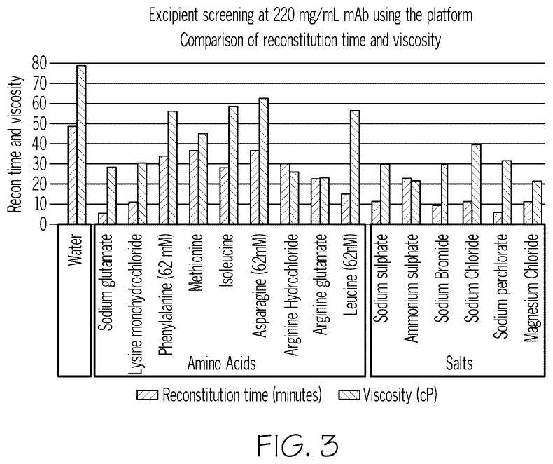

The protocol of Example 1 was implemented to screen different viscosity reducers and it was found that several salts and charged amino acids at 150 mM concentration reduced viscosity of reconstituted solution (190 or 220 mg/ml) to acceptable level (<30 cps) while preserving short reconstitution time and acceptable levels of HMW species. The reconstitution buffer comprises 10 mM histidine, 2% sucrose 150 mM excipient and 190 or 220 mg/ml mAb. The pH was 5.9. Results are depicted in FIG. 1 and FIG. 3.

### Example 3—Reconstitution Time, Viscosity and Δ% HMW

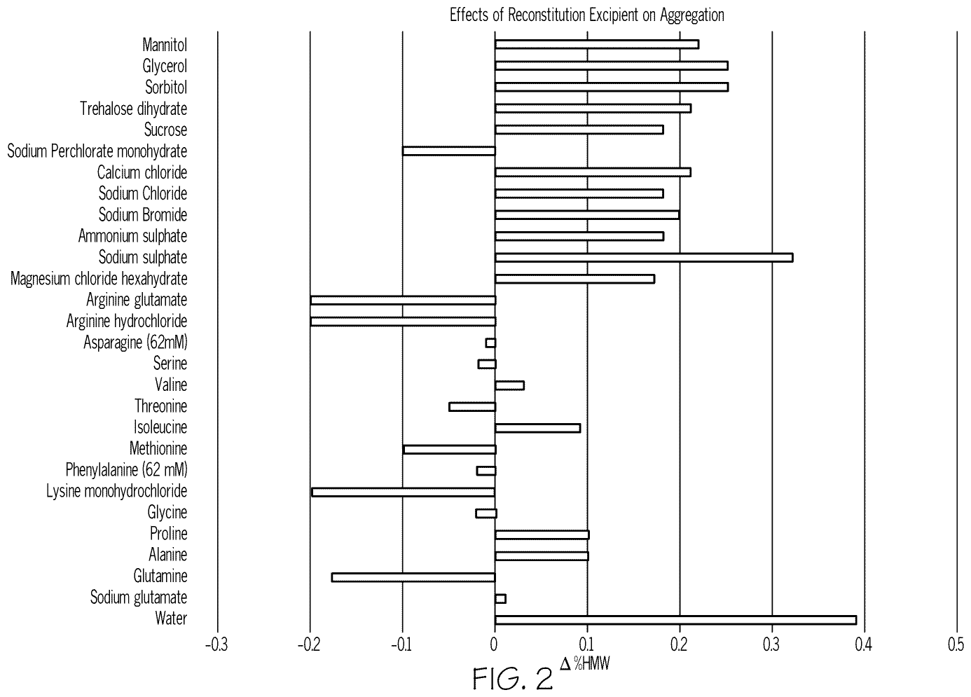

Table 2 discloses data from controlled nucleation and vacuum stoppering using monoclonal antibodies at concentration of 190 mg/ml and 220 mg/ml. Reconstitution time, viscosity, and changes in percentage of high molecular weight species was measured. Results show that changes in percentage of high molecular weight species (FIG. 2, Table 2) were modest for all tested excipients confirming usability of this screening method. Reconstitution time changes in the presence of excipients followed viscosity changes (FIGS. 1-2, Table 2).

TABLE 2

| | Reconstitution Time | | | | |
|---|---|---|---|---|---|
| | (minutes) | | Viscosity (cP) | | Δ% HMW |
| | Reconstitution Buffer | | | | |
| | 190 | 220 | 190 | 220 | 190 |
| | mg/mL | mg/mL | mg/mL | mg/mL | mg/m |
| | mAb | mAb | mAb | mAb | mAb |
| Water | 18 | 50 | 29 | 80 | 0.4 |
| 150 mM NaCl | 8 | 15 | 11 | 42 | 0.2 |
| 150 mM Arg- | 9 | 32 | 10 | 26 | 0.2 |
| HCl | | | | | |
| 150 mM Arg- | 11 | 23 | 11 | 23 | 0.2 |
| Glu | | | | | |

### Example 4—Excipient Characterizations

This example shows the characterization of many excipients. FIG. 2 depicts data showing the effects of reconstitution excipients on aggregation (Δ% HMW). Results show that changes in percentage of high molecular weight species were modest for all tested excipients confirming usability of this screening method. The results demonstrated that the lyophilization platform with minimum stabilizer (mAb to sucrose weight ratio 10:1) can be applied to screen high concentration mAb formulations for optimum compositions and pH with acceptable viscosity and stability.

FIG. 3 depicts data from excipient screening of a smaller number of amino acid excipients and salt excipients for a mAb preparation at 220 mg/ml. Reconstitution time and viscosity (cP) are depicted.

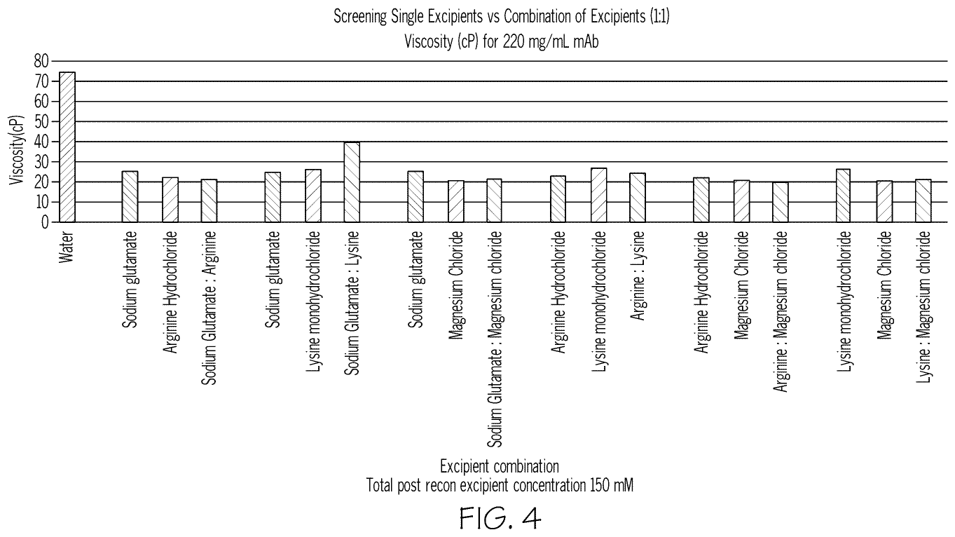

FIG. 4 depicts data from viscosity screening of single excipients vs combinations of excipients at a 1:1 ratio. The monoclonal antibody was present at 220 mg/ml.

FIG. 4 contains six groups. Each group shows data for each excipient separately and then each excipient combined at a 1 to 1 ratio (1:1). In some cases, cumulative effects of excipients on solution viscosity were observed. The results demonstrate that the platform can be applied to screen excipient combinations for high concentration mAb formulations.

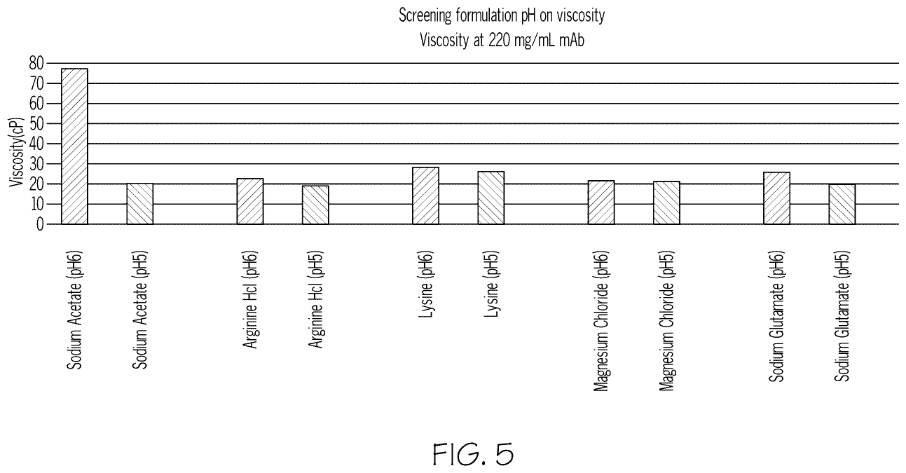

FIG. 5 depicts the effect of pH on viscosity (cP) using a monoclonal antibody present at a concentration of 220 mg/ml. Each excipient was tested at pH 5 or pH 6 to determine the effect on viscosity. In most cases viscosity was lower at pH 5 compared to pH 6. The results demonstrate that the platform can be applied to screen pH/buffer for high concentration mAb formulations.

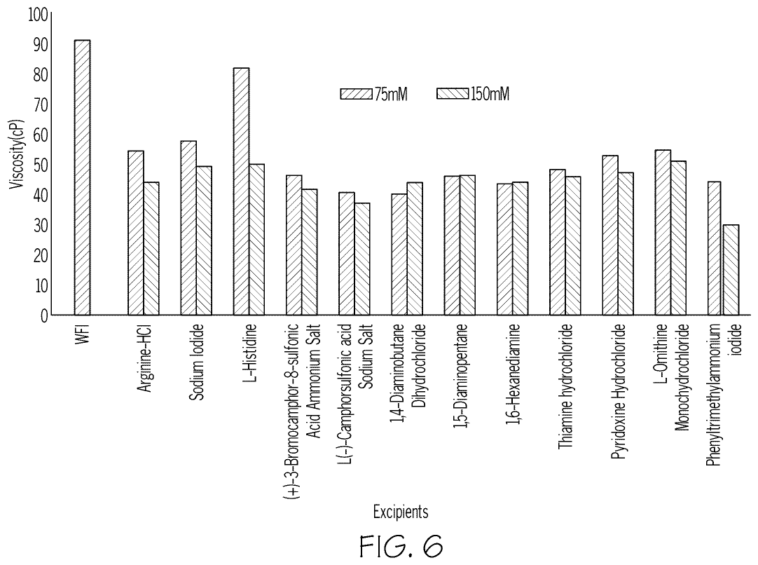

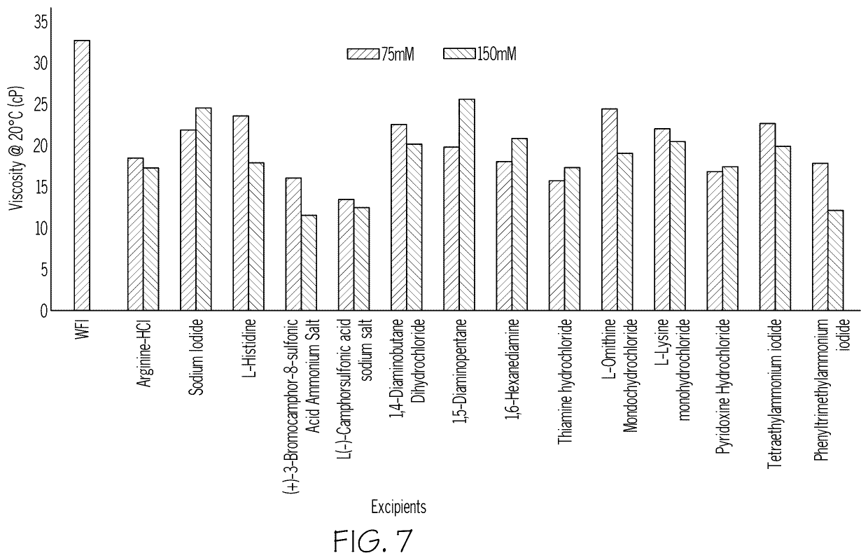

FIG. 6 is a bar graph depicting the viscosity of reconstituted solutions of a monoclonal antibody at a concentration of 250 mg/ml with potential viscosity reducing excipients at 75 mM and 150 mM. FIG. 7 is a bar graph depicting the viscosity of reconstituted solutions of a monoclonal antibody at a concentration of 200 mg/ml with potential viscosity reducing excipients at 75 mM and 150 mM. WFI stand for water for injection (no excipient).

FIG. 6 (250 mg/ml mAb) and FIG. 7 (200 mg/ml mAb) using excipients at 75 mM or 150 mM collectively show that some of the tested excipients (for example, camphorsulfonic acid and phenyltrimethylammonium iodide) result in a lower solution viscosity than arginine, which is a commonly used viscosity reducing excipient. The results demonstrate that the platform can be applied to screen excipient and excipient combinations for high concentration mAb formulations to find even more effective excipients or combinations than commonly used viscosity reducer (for example, arginine hydrochloride).

### Example 5—Exemplary Antibodies

According to additional aspects of the inventions, the antibody can be selected from the group consisting of an anti-Programmed Cell Death 1 antibody (for example an anti-PD1 antibody as described in U.S. Pat. Appln. Pub. No. US2015/0203579A1), an anti-Programmed Cell Death Ligand-1 (for example an anti-PD-L1 antibody as described in in U.S. Pat. Appln. Pub. No. US2015/0203580A1), an anti-DII4 antibody, an anti-Angiopoetin-2 antibody (for example an anti-ANG2 antibody as described in U.S. Pat. No. 9,402,898), an anti-Angiopoetin-Like 3 antibody (for example an anti-AngPtl3 antibody as described in U.S. Pat. No. 9,018,356), an anti-platelet derived growth factor receptor antibody (for example an anti-PDGFR antibody as described in U.S. Pat. No. 9,265,827), an anti-Erb3 antibody, an anti-Prolactin Receptor antibody (for example anti-PRLR antibody as described in U.S. Pat. No. 9,302,015), an anti-Complement 5 antibody (for example an 25 anti-C5 antibody as described in U.S. Pat. Appln. Pub. No US2015/0313194A1), an anti-TNF antibody, an anti-epidermal growth factor receptor antibody (for example an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (for example an anti-PCSK9 antibody as described in U.S. Pat. No. 8,062,640 or U.S. Pat. Appln. Pub. No. US2014/0044730A1), an anti-Growth And Differentiation Factor-8 antibody (for example an anti-GDF8 antibody, also known as anti-myostatin antibody, as described in U.S. Pat Nos. 8,871,209 or 9,260,515), an anti-Glucagon Receptor (for example anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g. an anti-IL4R antibody as described in U.S. Pat. Appln. Pub. No. US2014/0271681A1 or U.S. Pat Nos. 8,735,095 or 8,945,559), an anti-interleukin 6 receptor antibody (for example an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example anti-IL33 antibody as described in U.S. Pat. Appln. Pub. Nos. US2014/0271658A1 or US2014/0271642A1), an anti-Respiratory syncytial virus antibody (for example anti-RSV antibody as described in U.S. Pat. Appln. Pub. No. US2014/0271653A1), an anti-Cluster of differentiation 3 (for example an anti-CD3 antibody, as described in U.S. Pat. Appln. Pub. Nos. US2014/0088295A1 and U.S. Pat. No. 20150266966A1, and in U.S. Application No. 62/222,605), an anti-Cluster of differentiation 20 (for example an anti-CD20 antibody as described in U.S. Pat. Appln. Pub. Nos. US2014/0088295A1 and U.S. Pat. No. 20150266966A1, and in U.S. Pat. No. 7,879,984), an anti-CD19 antibody, an anti-CD28 antibody, an anti-Cluster of Differentiation 48 (for example anti-CD48 antibody as described in U.S. Pat. No. 9,228,014), an anti-Fel d1 antibody (for example as described in U.S. Pat. No. 9,079,948), an anti-Middle East Respiratory Syndrome virus (for example an anti-MERS antibody as described in U.S. Pat. Appln. Pub. No. US2015/0337029A1), an anti-Ebola virus antibody (for example as described in U.S.

Pat. Appln. Pub. No. US2016/0215040), an anti-Zika virus antibody, an anti-Lymphocyte Activation Gene 3 antibody (for example an anti-LAG3 antibody, or an anti-CD223 antibody), an anti-Nerve Growth Factor antibody (for example an anti-NGF antibody as described in U.S. Pat. Appln. Pub. No. US2016/0017029 and U.S. Pat. Nos. 8,309,088 and 9,353,176) and an anti-Activin A antibody. In some embodiments, the bispecific antibody is selected from the group consisting of an anti-CD3 x anti-CD20 bispecific antibody (as described in U.S. Pat. Appln. Pub. Nos. US2014/0088295A1 and U.S. Pat. No. 20150266966A1), an anti-CD3 x anti-Mucin 16 bispecific antibody (for example, an anti-CD3 x anti-Muc16 bispecific antibody), and an anti-CD3 x anti-Prostate-specific membrane antigen bispecific antibody (for example, an anti-CD3 x anti-PSMA bispecific antibody). See also U.S. Patent Publication No. US 2019/0285580 A1. Also included are a Met x Met antibody, an agonist antibody to NPR1, an LEPR agonist antibody, a BCMA x CD3 antibody, a MUC16 x CD28 antibody, a GITR antibody, an IL-2Rg antibody, an EGFR x CD28 antibody, a Factor XI antibody, antibodies against SARS-CoC-2 variants, a Fel d 1 multi-antibody therapy, a Bet v 1 multi-antibody therapy. Derivatives, components, domains, chains and fragments of the above also are included.

Cells that produce exemplary antibodies can be cultured according to the inventions. Exemplary antibodies include Alirocumab, Atoltivimab, Maftivimab, Odesivimab, Odesivivmab-ebgn, Casirivimab, Imdevimab, Cemiplimab and Cemiplimab-rwlc (human IgG4 monoclonal antibody that binds PD-1), Dupilumab (human monoclonal antibody of the IgG4 subclass that binds to the IL-4R alpha (a) subunit and thereby inhibits Interleukin 4 (IL-4) and Interleukin 13 (IL-13) signalling), Evinacumab, Evinacumab-dgnb, Fasinumab, Fianlimab, Garetosmab, Itepekimab Nesvacumab, Odrononextamab, Pozelimab, Sarilumab, Trevogrumab, and Rinucumab.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

It is to be understood that the description, specific examples and data are given by way of illustration and are not intended to limit the present inventions. Various changes and modifications within the present inventions, including combining teaching in whole and in part, will become apparent to the skilled artisan from the discussion, disclosure and data contained herein, and thus are considered part of the inventions.

What is claimed is:

1. A method for selecting excipients for inclusion in a high concentration Fc-containing protein formulation, wherein the method comprises the steps of:
(a) combining at least one excipient with a preparation comprising an Fc-containing protein;
(b) conducting a controlled nucleating cycle on the preparation from step (a);
(c) drying the preparation from step (b) under a vacuum;
(d) stoppering with and/or without a vacuum; and
(e) evaluating excipient performance for use in a Fc-containing protein formulation.

2. The method according to claim 1, wherein secondary drying is not employed.

3. The method according to claims 1-2, wherein the excipient is selected from the group consisting of amino acids, salts, polyols and combinations thereof.

4. The method according to claims 1-3, wherein the excipient is a polyol selected from the group consisting of sucrose, trehalose dihydrate, sorbitol, glycerol and combinations thereof.

5. The method according to claims 1-4, wherein the excipient is a salt selected from the group consisting magnesium chloride, sodium sulphate, ammonium sulphate, sodium bromide, sodium chloride, calcium chloride, sodium perchlorate and combinations thereof.

6. The method according to claim 1-5, wherein the excipient is an amino acid selected from the group consisting of sodium glutamate, glutamate, alanine, proline, glycine, lysine, phenylalanine, methionine, isoleucine, threonine, valine, serine, asparagine, histidine arginine hydrochloride, arginine glutamate, and combinations thereof.

7. The method according to claims 1-6, wherein the Fc-containing protein is a monoclonal antibody.

8. The method according to claims 1-7, wherein the Fc-containing protein formulation comprises at least one excipient selected from the group consisting of
(i) at least one polyol selected from the group consisting of sucrose, trehalose dihydrate, sorbitol, glycerol and combinations thereof;
(ii) at least one salt selected from the group consisting magnesium chloride, sodium sulphate, ammonium sulphate, sodium bromide, sodium chloride, calcium chloride, sodium perchlorate and combinations thereof; and
(iii) at least one amino acid selected from the group consisting of sodium glutamate, glutamate, alanine, proline, glycine, lysine, phenylalanine, methionine, isoleucine, threonine, valine, serine, asparagine, histidine arginine hydrochloride, arginine glutamate, and combinations thereof.

9. The method according to claims 1-8, wherein the Fc-containing protein formulation comprises a salt and an amino acid as excipients.

10. The method according to claim 1-8, wherein the Fc-containing protein formulation comprises a salt and a polyol as excipients.

11. The method according to claims 1-8, wherein the Fc-containing protein formulation comprises an amino acid and a polyol as excipients.

12. The method according to claims 1-8, wherein the Fc-containing protein formulation comprises an amino acid, a salt and a polyol as excipients.

13. An Fc-containing protein formulation comprising one or more excipients selected according to the methods of claims 1-12.

14. The Fc-containing formulation of claim 13, wherein the Fc-containing protein is a monoclonal antibody.
